(19) United States
(12) Patent Application Publication
(10) Pub. No.: US 2015/0056175 A1
(43) Pub. Date: Feb. 26, 2015

(54) COMPOSITION FOR PREVENTING AND/OR TREATING DERMATOSIS AND METHOD FOR OBTAINING SAME

(71) Applicant: Juan Miguel Garcia Gilabert, Puerto De Mazarron (ES)

(72) Inventor: Juan Miguel Garcia Gilabert, Puerto De Mazarron (ES)

(21) Appl. No.: 14/386,547

(22) PCT Filed: Mar. 16, 2013
(86) PCT No.: PCT/ES2013/070178

§ 371 Date: Sep. 19, 2014

(30) Foreign Application Priority Data
Mar. 22, 2012 (ES) P201230434

(51) Int. Cl. A61K 9/00; A61K 36/28; A61K 36/53; A61K 36/54; A61K 36/185; A61K 36/899; A61K 36/06; A61K 36/76; A61K 36/48; A61K 36/63; A61K 36/886; A61K 36/8998; A61K 36/47; A61K 36/14; A61K 35/36; A61K 36/77; A61K 36/30; A61K 35/64

(52) U.S. Cl. CPC A61K 9/0014; A61K 35/644; A61K 36/28; A61K 36/53; A61K 36/54; A61K 36/185; A61K 36/899; A61K 36/06; A61K 36/76; A61K 36/48; A61K 36/63; A61K 36/886; A61K 36/8998; A61K 36/47; A61K 36/14; A61K 35/36; A61K 36/77; A61K 36/30
USPC 424/93.51

(57) ABSTRACT

A composition for preventing and/or treating dematosis, in particular psoriasis, and to a method for obtaining the same, which includes chamomile, thyme, bay leaf, hops, millet, barley malt, brewer's yeast, willow, fenugreek (Trigonella foenum-graecum), salicaria (Lythrum salicaria), hibiscus (Hibiscus), olive oil, bee's honey, aloe vera, oat bran, chicory, coparchy (Coutarea latifolia), Stevia, wheat flour, cypress, brewer's malt, lanolin, elderberry, goldenrod (Solidago), witch-hazel, heal-all (Prunella vulgaris), and St John's wort (Hypericum perforatum).

### PURPOSE OF THE INVENTION

The present invention relates to a composition for treating and preventing dermatosis, whether recent or old, and in particular psoriasis.

### BACKGROUND OF THE INVENTION

Abnormal scaly and/or keratotic formations of the dermis are manifestations of the condition of the skin that are encountered in many dermatoses, whether they are of recent appearance or are very old, in particular the various forms of psoriasis.

Psoriasis is a chronic progressive erythematosquamous dermatosis that comprises a plurality of red plaques covered with thick white scales and affects 2% of the population. With more than 60000 new persons affected each year, it is one of the commonest dermatological disorders. This disorder is manifested by painful stinging and lesions that may cover more than 10% of the body. In its usual forms, its localization is very characteristic: elbows and cubital side of the forearms, knees, lumbosacral region, scalp and nails.

Some particular forms, described as severe psoriasis, are also known, such as erythrodermic psoriasis, psoriasis arthropathica or pustular psoriasis. Approximately 1.5% of psoriasis cases begin in childhood before the age of 10 years and 35% before the age of 20 years. This abnormality of renewal of the epidermis is therefore a serious disorder that represents an important public health problem.

The cause of psoriasis is unknown at present, but it is believed that multiple factors are involved. It has been possible to demonstrate a genetic factor, with 30% of psoriasis cases being familial, as well as some environmental factors. Psychological factors may often initiate some attacks. Skin injuries, alcohol and tobacco are some factors in severity and therapeutic resistance.

The evolution of the disease is chronic and occurs as attacks that are generally unforeseeable, interrupted by remissions during which the lesions are minimal. Even apart from the severe forms, and even though the patient's general condition is unchanged in the usual forms, psoriasis is a disease that may have profound effects on quality of life when the lesions are visible or uncomfortable for manual work, consequences that are often underestimated by the physician.

At present, physicians have at their disposal symptomatic treatments with temporary results, but not yet any effective medicinal products.

### DESCRIPTION OF THE INVENTION

The aim of the present invention is to propose a composition for treating states caused by or that are manifested as abnormal keratotic and/or scaly formations of the dermis, such as those that are encountered in recent or chronic dermatosis and, in particular, in the various forms of psoriasis.

In a first aspect, the present invention relates to a composition for treating dermatosis (hereinafter composition of the present invention), characterized in that it comprises chamomile, thyme, laurel, hops, millet, barley malt, brewer's yeast, willow, fenugreek (Trigonella foenum-graecum), purple loosestrife (Lythrum salicaria); hibiscus (Hibiscus), olive oil, honey, aloe vera, oat bran, chicory, copalchi (Coutarea latifolia), Stevia, wheat flour, cypress, brewing malt, lanolin, elder, goldenrod (Solidago), Hamamelis; comfrey (Prunella vulgaris), and St. John's wort (Hypericum perforatum).

In a second aspect, the present invention relates to a method for obtaining the composition of the present invention, characterized in that it comprises grinding all the elements, except the aloe vera; then all the elements previously ground are diluted in water, leaving it to boil and to liquefy after it has boiled; and in that the aloe vera, on the other hand, is liquefied directly and is then mixed with the products previously boiled and liquefied, and the new mixture is liquefied again, leaving it to ferment in an open container prior to packaging.

Finally, it should be pointed out that the composition is especially useful for the topical treatment of psoriasis, but is also applicable for: spots on the skin, pimples, cellular regeneration, skin sweating and odors, skin tightening agent, chafing from footwear or some other type of chafing, antiwrinkle agents, acne, wound healing, eczemas, blackheads, hemorrhoids, burns, skin ulcers, chilblains, hematomas, insect bites, skin care, wounds and shaver's rash.

### PREFERRED EMBODIMENT OF THE INVENTION AND EXAMPLES OF USE

In a preferred embodiment of the invention, the composition for preventing and/or treating dermatosis comprises diluting the following products in 40 liters of water: 150 g of chamomile; 150 g of thyme; 150 g of laurel; 150 g of hops; 150 g of millet; 200 g of barley malt; 150 g of brewer's yeast; 150 g of willow; 150 g of fenugreek (Trigonella foenum-graecum); 150 g of purple loosestrife (Lythrum salicaria); 200 g of hibiscus (Hibiscus); 1.5 liters of olive oil; 1 kg of honey; 5 kilos of aloe vera; 300 g of oat bran; 150 g of chicory; 150 g of copalchi (Coutarea latifolia); 200 g of Stevia; 1 kg of wheat flour; 150 g of cypress; 200 g of brewing malt; 150 cm³ of lanolin; 150 g of elder; 150 g of goldenrod (Solidago); 150 g of Hamamelis; 150 g of comfrey (Prunella vulgaris); and 150 g of St. John's wort (Hypericum perforatum).

The method of preparation is as follows:

In a first step, all the elements are ground, except the aloe vera. Then all the elements previously ground are diluted in boiling water (40 liters), for a period of time of between 10 and 12 minutes. Once the products have been boiled, they liquefy.

The aloe vera, on the other hand, is liquefied directly and is then mixed with the products previously boiled and liquefied, and the new mixture is liquefied again. The mixture is fermented for 3-4 days in an open container, prior to packaging.

The application or use of the product starts from topical application thereof on the affected zone, daily and for a period of between 2 and 3 months, noting improvement of the affected zone from ten or twelve days after application until complete disappearance of the scabs and scales typical of psoriasis toward the end of said period of application, observing complete disappearance of said scabs and scales and persistence of said disappearance, no reappearance of scabs and/or scales occurring in trials during the year following the end of treatment.

1. A composition for treating dermatosis that comprises chamomile, thyme, laurel, hops, millet, barley malt, brewer's yeast, willow, fenugreek (Trigonella foenum-graecum), purple loosestrife (Lythrum salicaria); hibiscus (Hibiscus), olive oil, honey, aloe vera, oat bran, chicory, copalchi (Coutarea latifolia), Stevia, wheat flour, cypress, brewing malt, lanolin, elder, goldenrod (Solidago), Hamamelis; comfrey (Prunella vulgaris), and St. John's wort (Hypericum perforatum).

2. The composition as claimed in claim 1 blended to a liquefied state suitable for treating psoriasis by topical application.

3. A method for forming a composition as claimed in claim 1 for treating dermatosis comprising grinding all the elements, except the aloe vera; then all the elements previously ground are diluted in water, leaving it to boil and to liquefy after it has boiled; and in that the aloe vera, on the other hand, is liquefied directly and is then mixed with the products previously boiled and liquefied, and the new mixture is liquefied again, leaving it to ferment in an open container prior to packaging.

4. A method of making a medicinal product for treating psoriasis by topical application, comprising:
blending the composition of claim 1 into a liquefied state; and
packaging an amount of the blended composition.

5. A method for preventing psoriasis by topical application, comprising:
providing a medicinal product comprised of chamomile, thyme, laurel, hops, millet, barley malt, brewer's yeast, willow, fenugreek (Trigonella foenum-graecum), purple loosestrife (Lythrum salicaria); hibiscus (Hibiscus), olive oil, honey, aloe vera, oat bran, chicory, copalchi (Coutarea latifolia), Stevia, wheat flour, cypress, brewing malt, lanolin, elder, goldenrod (Solidago), Hamamelis; comfrey (Prunella vulgaris), and St. John's wort (Hypericum perforatum), and
topically applying an effective amount of the medicinal product to an affected zone of the skin of a person to be treated.

6. The method of claim 5 further comprising: topically applying the medicinal product daily for a period of 2 months.

7. The method of claim 4 wherein blending step further comprises:
blending together all of the ingredients except aloe vera and boiling the blended ingredients in water until liquefied;
mixing with the blended ingredients after boiling with the aloe vera until the combined mixture is again liquefied; and
fermenting the resulting combined mixture prior to packaging.
